(19) United States
(12) Patent Application Publication
(10) Pub. No.: US 2012/0145162 A1
(43) Pub. Date: Jun. 14, 2012

(54) ETT HOLDER

(76) Inventors: Thomas R. Haddix, Monroe, MI (US); Steven J. Haddix, Allen Park, MI (US)

(21) Appl. No.: 13/315,600

(22) Filed: Dec. 9, 2011

Related U.S. Application Data
(60) Provisional application No. 61/421,899, filed on Dec. 10, 2010.
(60) Provisional application No. 61/491,879, filed on May 31, 2011.

(51) Int. Cl. A61M 16/04

(52) U.S. Cl. 128/207.17; 128/207.14

(57) ABSTRACT

An intra-oral device for positioning an endotracheal tube in the mouth of a patient includes a block disposable between the patients upper and lower teeth. The block has a top surface including an arcuate channel for receiving upper teeth. The block also has a bottom surface including an arcuate channel for receiving lower teeth. The block has an outwardly facing, when disposed, front surface and also a back surface and an aperture extending through the block from the front to the back surfaces for receiving the endotracheal tube. A slot having parallel spaced walls extends from the bottom surface to the aperture and from the front surface to the back surface. A cincher is disposed to cinch the spaced walls of the slot. An endotracheal tube inserted through the aperture can be positioned and cinched in the device.

### CROSS REFERENCE TO RELATED APPLICATIONS

This application claims the priority of U.S. Provisional Application No. 61/421,899 filed Dec. 10, 2010 and U.S. Provisional Application No. 61/491,879 filed May 31, 2011.

### TECHNICAL FIELD

This invention relates to tracheal intubation, and more particularly to securement of an endotracheal tube (ETT) on a patient after intubation.

### BACKGROUND OF THE INVENTION

It is known in the art relating to tracheal intubation that after an endotracheal tube (ETT) has been inserted through a patient's mouth and vocal chords into the trachea, the ETT may be secured directly to the patient's face or neck with one or more strips of medical tape to prevent the ETT from moving. However, the adhesive on the tape can irritate the skin, especially if the patient has thin and or sensitive skin. The irritation caused by the tape may undesirably lead to painful and unsightly sores, cuts, and abrasions on the patient's face, neck, and ears where the tape is disposed.

### SUMMARY OF THE INVENTION

The present invention provides an endotracheal tube (ETT) holder that secures an ETT while the tube is inserted in a patient's trachea during surgery or when it is otherwise medically necessary to maintain an open airway in the patient. The present ETT holder eliminates the need to use tape to secure the tube to a patient's face, thereby preventing sores, cuts, and abrasions from developing on the patient's skin. The present ETT holder is easier to use than tape strips for the securement of the tube on the patient. In addition, the present ETT holder facilitates the suctioning of the mouth and throat while the endotracheal tube is in place.

More particularly, an intra-oral device for positioning an endotracheal tube in the mouth of a patient includes a block disposable between the patient's upper and lower teeth. The block has a top surface including an arcuate channel for receiving upper teeth. The block also has a bottom surface including an arcuate channel for receiving lower teeth. The block has an outwardly facing, when disposed, front surface and also a back surface and an aperture extending through the block from the front to the back surfaces for receiving the endotracheal tube. A slot having parallel spaced walls extends from the bottom surface to the aperture and from the front surface to the back surface. A cincher is disposed to cinch the spaced walls of the slot. An endotracheal tube inserted through the aperture can be positioned and cinched in the device.

The intra-oral device may include side surfaces that mirror each other and taper inwardly from said front to said back surfaces. The side surfaces may be spaced to fit within the width of the patient's mandible. The side surfaces each may also include an attachment member adjacent the front surface. The intra-oral device also may include a head fastening strap attachable to the attachment members. The cincher may include an elongated strip having a plurality of apertures and a pair of knobbed posts extending from the bottom surface of the block. The plurality of apertures in the strip are cooperable with the knobbed posts.

These and other features and advantages of the invention will be more fully understood from the following detailed description of the invention taken together with the accompanying drawings.

### BRIEF DESCRIPTION OF THE DRAWINGS

In the drawings:

FIG. 1 is a perspective view of an intra-oral device in accordance with the present invention;

FIG. 2 is a front view of the intra-oral device;

FIG. 3 is a side view of the intra-oral device;

FIG. 4 is a plan view of the intra-oral device; and

FIG. 5 is an environmental view of the intra-oral device securing an endotracheal tube in a patient's mouth.

### DETAILED DESCRIPTION OF THE INVENTION

Referring now to the drawings in detail, numeral 10 generally indicates an intra-oral device (ETT holder) in accordance with the present invention for positioning an endotracheal tube (ETT) in the mouth of a patient. The intra-oral device 10 is simple to install and easily secures an ETT in a patient's mouth. The intra-oral device 10 also may be secured about a patient's face without the use of adhesive tape, thereby reducing the patient's exposure to irritating adhesives. The intra-oral device 10 also prevents a patient from biting down on the ETT, which can block the airway formed through the ETT. Further, the intra-oral device 10 is narrower than the patient's jaws and holds the patient's mouth slightly open to allow for the suctioning of fluid from the patient's mouth and throat while the ETT is in place.

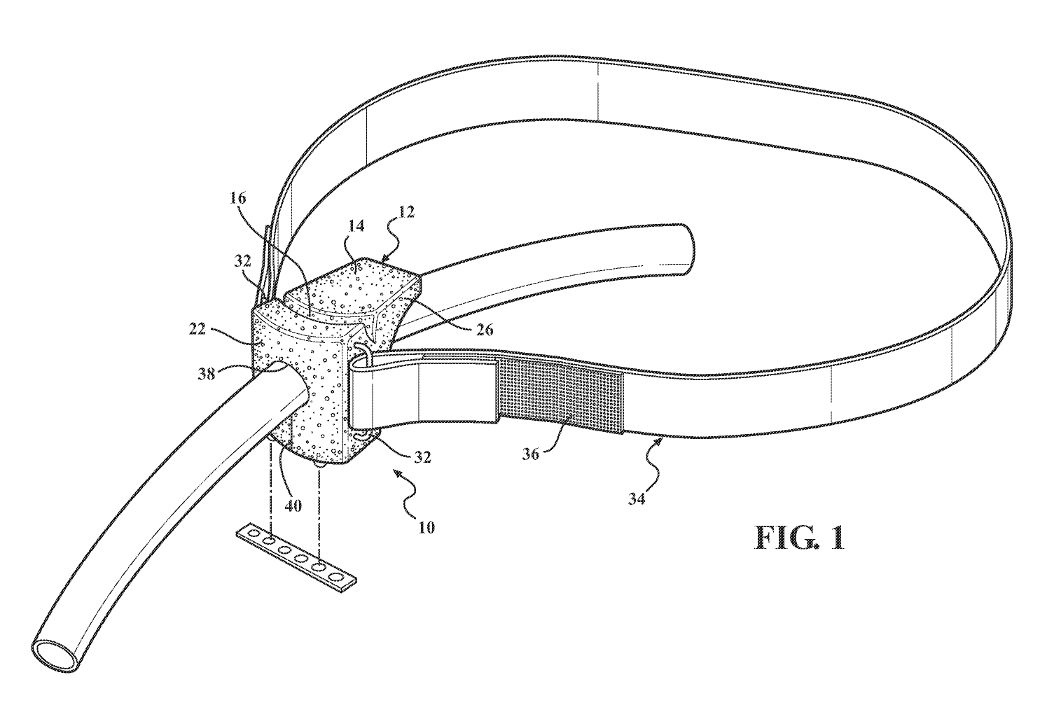

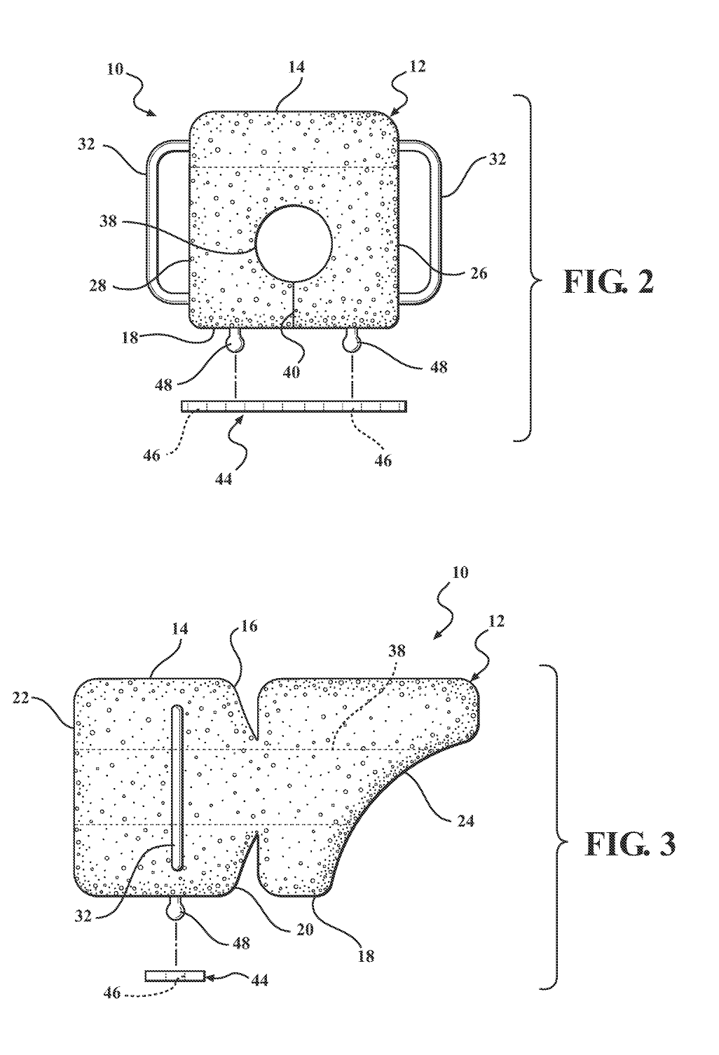

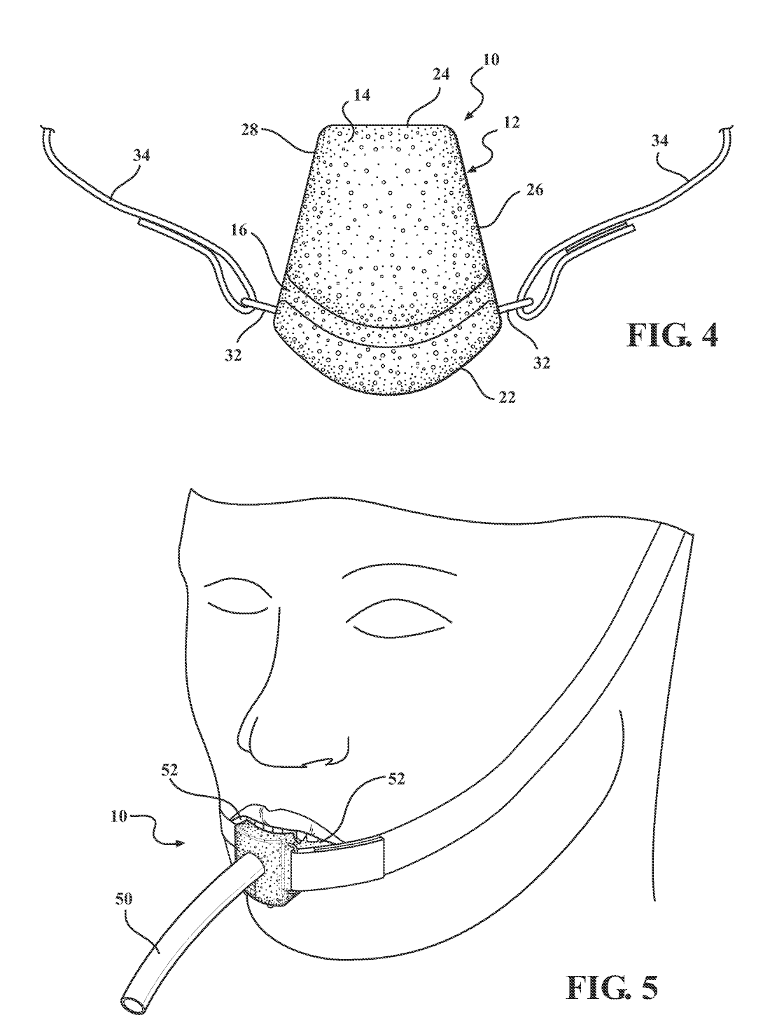

As shown in FIGS. 1 through 4, the intra-oral device 10 includes a block 12 disposable between the patient's upper and lower teeth. The block 12 may be formed of a foam rubber material or other similar material suitable for medical use. The block 12 has a top surface 14 including an arcuate channel for receiving upper teeth of the patient. The block 12 also has a bottom surface 18 including an arcuate channel 20 for receiving lower teeth of the patient. The block 12 has an outwardly facing, when disposed in a patient's mouth, front surface 22 and an opposite back surface 24. The block also includes side surfaces 26, 28 that mirror each other and that taper inwardly from the front surface 22 to the back surface 24. The side surfaces 26, 28 may be spaced to fit within the width of the patient's mandible, i.e. the device 10 is narrower than the width of the mandible. As discussed in more detail below, the device 10 therefore does not block and/or interfere with access to the patient's mouth, allowing a separate suction tube to be inserted along the side of the patient's mouth.

The side surfaces 26, 28 each include an attachment member 32 adjacent the front surface 22. A head fastening strap 34 is attachable to the attachment members 32. The strap 34 may include a hook-and-loop fastener 36 that allows for opening and closing of the strap as well as adjustment of the length of the strap. The strap 32 may be wrapped around a patient's head, allowing the device 10 to be releasably secured to the patient's face (without tape) in addition to or in the alternative to the dental adhesive 30.

An aperture 38 extends through the block 12 from the front surface 22 to the back surface 24 for receiving an endotracheal tube therethrough. A slot 40 having parallel spaced walls 42 extends from the bottom surface 18 to the aperture 26 and from the front surface 22 to the back surface 24. An endotracheal tube may inserted into the aperture 38 through the slot 40, which allows the device 10 to be placed on the tube at any location along the length of the tube. A cincher 44 is disposed to cinch the spaced walls 42 of the slot 40. The cincher 44 may be formed of an elongated, flexible strip having a plurality of spaced apertures 46 through which knobbed posts 48 extending from the bottom surface 18 of the block 12 may be inserted. An endotracheal tube inserted through the aperture 38 can be positioned and cinched in the device 10.

Turning now to FIGS. 1 through 5, the device 10 is shown securing an endotracheal tube 50 that is inserted through a patient's mouth 52. Prior to securement in the device 10, the endotracheal tube 50 is intubated into the patient's trachea to provide an airway to the patient's lungs. Once intubation of the endotracheal tube 50 is completed, the device 10 is installed to hold the endotracheal tube precisely in place. The device 10 is placed on the endotracheal tube 50 by either feeding the outer end of the tube through the aperture 38, or more preferably by inserting the tube sideways into the aperture through the slot 40. The cincher 44 is then secured to pull the spaced walls 42 of the slot 40 towards one another and to tighten the aperture 38 around the tube 50 to prevent slipping of the tube in the slot. Next, the device 10 is mounted on the patient by manipulating the device so that the patient's upper and lower teeth are disposed in the top and bottom arcuate channels 16, 20, respectively. The strap 34 is looped around the patient's head and tightened to further secure the device 10 in place. Thus, the endotracheal tube 50 can remain inserted in and secured to the patient for extended periods of time without any damage to the patient's skin from taping.

The device 10 also prevents the patient from biting down on the endotracheal tube 50. The patient's teeth bite into the arcuate channels 16, 20 without affecting (i.e., compressing) the tube 46 that is disposed through the aperture 38. Due to the space between the top arcuate channel 16 and the bottom arcuate channel 18, when a patient bites the device 10, a gap will exist between the patient's upper and lower teeth. Also, the device 10 is narrower than the patient's mandible. Therefore, when the device 10 is mounted in the patient's mouth, a gap exists between the device and the corners of the patient's mouth on each side of the device. The gaps allow for the insertion of a separate suction tube for periodically removing fluid from the patient's mouth and throat.

Although the invention has been described by reference to a specific embodiment, it should be understood that numerous changes may be made within the spirit and scope of the inventive concepts described. Accordingly, it is intended that the invention not be limited to the described embodiment, but that it have the full scope defined by the language of the following claims.

What is claimed is:

1. An intra-oral device for positioning an endotracheal tube in the mouth of a patient, the device comprising:
a block disposable between the patient's upper and lower teeth;
said block having a top surface including an arcuate channel for receiving upper teeth;
said block also having a bottom surface including an arcuate channel for receiving lower teeth;
said block having an outwardly facing, when disposed, front surface and also a back surface and an aperture extending through said block from said front to said back surfaces for receiving the endotracheal tube;
a slot having parallel spaced walls extending from said bottom surface to said aperture and from said front surface to said back surface;
a cincher disposed to cinch said spaced walls of said slot;
whereby an endotracheal tube inserted through said aperture can be positioned and cinched in said device.

2. The intra-oral device of claim 1 including side surfaces that mirror each other and taper inwardly from said front to said back surfaces.

3. The intra-oral device of claim 2 wherein said side surfaces are spaced to fit within the width of the patient's mandible.

4. The intra-oral device of claim 2 wherein said side surfaces each include an attachment member adjacent said front surface.

5. The intra-oral device of claim 4 including a head fastening strap attachable to said attachment members.

6. The intra-oral device of claim 1 wherein said cincher includes an elongated strip having a plurality of apertures and a pair of knobbed posts extending from the bottom surface of said block, said plurality of apertures being cooperable with said knobbed posts.
